(19) United States
(12) Patent Application Publication
(10) Pub. No.: US 2009/0318780 A1
(43) Pub. Date: Dec. 24, 2009

(54) APPARATUS AND METHOD FOR PROCESSING RESPIRATORY AIR TEMPERATURE AND PRESSURE INFORMATION

(75) Inventor: Peter Stasz, Moundsview, MN (US)

Correspondence Address: NIKOLAI & MERSEREAU, P.A., 900 SECOND AVENUE SOUTH, SUITE 820, MINNEAPOLIS, MN, 55402, US

(73) Assignee: Dymedix Corporation, Shoreview, MN (US)

(21) Appl. No.: 12/491,058

(22) Filed: Jun. 24, 2009

Related U.S. Application Data
(60) Provisional application No. 61/075,124, filed on Jun. 24, 2008.

(51) Int. Cl. A61B 5/08; G06F 15/00; G06F 19/00; G01L 7/00

(52) U.S. Cl. 600/301; 702/130; 702/50; 702/199

(57) ABSTRACT

An apparatus or method can be configured to receive information indicative of respiratory air temperature of a subject from a respiratory air temperature piezoelectric film sensor and information indicative of respiratory air pressure of the subject from a respiratory air pressure piezoelectric film sensor, and to simultaneously process the received respiratory air temperature information and the received respiratory air pressure information to produce a first electronic signal output indicative of respiratory air temperature and a second electronic signal output indicative of respiratory air pressure.

### CLAIM OF PRIORITY

This patent application claims the benefit of priority to U.S. Provisional Patent Application Ser. No. 61/075,124, filed on Jun. 24, 2008, which application is herein incorporated by reference in its entirety.

### TECHNICAL FIELD

This invention relates generally to an electronic signal processing circuit for adapting a polarized respiratory air temperature and pressure change sensor to a conventional polysomnograph (PSG) machine of the type commonly used in sleep laboratory applications, and more particularly to an adapter that simultaneously processes two separate polyvinylidene (PVDF) film transducer signals. More specifically one signal from a temperature change sensing PVDF film transducer and another signal from a pressure change sensing PVDF film transducer signal.

### BACKGROUND

In addressing sleep related problems, such as sleep apnea, insomnia and other physiologic events or conditions occurring during sleep, various hospitals and clinics have established laboratories sometimes referred to as “Sleep Laboratories” (sleep labs). At these sleep labs, using instrumentation, such as patient bio-data sensors connected to a polysomnograph (PSG) machine, a patient's sleep patterns may be monitored and recorded for later analysis so that a proper diagnosis may be made and a therapy prescribed. Varieties of sensors have been devised for providing recordable signals related to respiratory (inhaling and exhaling) patterns during sleep. These sensors commonly are mechanical to electrical transducers that produce an electrical signal related to respiration.

Current practice and procedure in sleep labs use two different sensing systems at the same time on the same patient that measure respiratory air temperature and pressure. Respiratory air temperature is typically measured using either a thermocouple or a thermistor attached directly to a sleep labs PSG machine. Respiratory air pressure is often measured using a nasal pressure prong cannula placed in the patient's nostrils and attached via a plastic hose to an air pressure transducer. The output of air pressure transducers connect directly to PSG machines.

Air pressure transducers with nasal cannulas in combination with either a thermistor or thermocouple, as used in sleep studies, are invasive, uncomfortable and prone to clogging and body movement and thus put an unnecessary strain and discomfort on the patients.

### SUMMARY

The present inventor has recognized, among other things, that there is a need to there is a need to provide an apparatus and method for processing respiratory air temperature and pressure change transducer signals that does not require the patient to wear two different and separate temperature and pressure sensors apparatus at the same time in nearly the same space.

Furthermore, there is a need for an apparatus and method for simultaneously processing respiratory air temperature and pressure change transducer signals in order to provide a rigid phase and polarity relationship between respiratory air temperature and pressure to final graphical indication of the individually processed PVDF film transducers signals on the PSG machine display.

Furthermore, there is a need to provide an apparatus and method for processing respiratory air temperature and pressure change transducer signals in order to provide the sleep professional with a simpler method to diagnose sleep related disorders.

Furthermore, there is also a need to provide an apparatus and method for processing respiratory air temperature and pressure change transducer signals in order to provide a rigid phase relationship between respiratory airflow (inspiration and expiration) to final graphical indication of the polarized piezoelectric film sensor signals on the PSG machine display.

In certain examples, a PVDF film can have both pyroelectric and piezoelectric properties and, as such, can be responsive to both inspiratory and expiratory air temperature and air pressure changes, producing a corresponding polarized electrical signal output indicating either inspiratory air temperature and pressure or expiratory air temperature and pressure. The polarized electrical sensor output signal can be processed to effectively separate the inspiratory or expiratory temperature change induced signal from the signal due to inspiratory or expiratory pressure change.

The present inventor has recognized, among other things, that an apparatus and method can be provided for processing respiratory air temperature and pressure change transducer signals for a polarized respiratory air temperature and pressure change sensor especially constructed to simultaneously detect respiratory air temperature and pressure changes at the same time with a single sensor for the comfort of a patient undergoing evaluation.

In certain examples, an adaptor can be provided for simultaneously processing the signals of a temperature change sensing PVDF film transducer and a pressure change sensing PVDF film transducer for further connection to a PSG machine.

In an example, the adapter can include two independent sets of differential amplifier and integrator circuits with resistive reset having a pair of input terminals that are adapted to be coupled to the outputs of a temperature change sensing PVDF film transducer and a pressure change sensing PVDF film transducer the polarized piezoelectric film sensor. The differential amplifier and signal integrator with resistive reset can be configured to provide a predetermined gain factor to amplify the temperature change sensing and the pressure change sensing PVDF film transducer output signals, to provide transducer signal averaging over time to reduce unwanted differential noise, and to significantly attenuate common-mode noise. Each of the outputs of the two differential amplifier and signal integrator with resistive reset circuits can be fed to a set of signal output attenuators.

By utilizing a differential input amplifier with a predetermined gain factor and by appropriately conditioning the amplified temperature change sensing and pressure change sensing PVDF film transducer output signals, the resulting filter outputs can be readily matched to existing PSG machines already on hand in most sleep laboratories.

The temperature change sensing and the pressure change sensing PVDF film transducers that are part of a respiratory airflow temperature and pressure change sensor can sense thermal and mechanical energies well in the GHz range. In various examples, because the normal human adult resting respiration rate can be relatively slow (e.g., 12 to 20 breaths per minute), irregular in frequency and amplitude and in a distorted sinusoidal form, the raw PVDF film transducer signals can be averaged over time to condition the signal for graphical presentation in the PSG machine. Sensor signal averaging over time can remove undesired patient motion signals and other environmentally induced noise signal artifacts.

Polarized lead wires can be provided to interface the PVDF film transducers and the PSG machine. One wire may be outfitted with a red marking and is designated positive. The other wire may be outfitted with a black marking and is designated negative. The PVDF film transducer can generate a direct current (DC) voltage, much like a battery, when subjected to temperature and pressure variations during exhaling and inhaling. During respiration, the temperature sensing PVDF film transducer can be subjected to a change in thermal energy of expired air molecules and the pressure sensing PVDF film transducer can be subjected to a change in kinetic energy of expired air molecules. Lead wires can be provided to interface between the sensor and the PSG machine. In an example, the positive designated PVDF film transducer surface electrode can become negatively charged when exhaling, and the positive designated PVDF film transducer surface electrode can become positively charged when inhaling. Accordingly, the negative designated PVDF film transducer surface electrode can become negatively charged when inhaling, and the negative designated PVDF film transducer surface electrode can become positively charged when exhaling.

In an example, when a negative voltage/charge is presented to the PSG input reference terminal, an upward deflection on the PSG display can indicate a respiratory exhalation effort. When a positive voltage/charge is presented to the PSG input reference terminal, a downward deflection on the PSG display can indicate a respiratory inhalation effort.

In order to maintain the polarity processing properties and to minimize potentially long phase delays, all electronic signal-processing paths can be DC coupled. Persons skilled in the art will recognize the DC coupling when reviewing the disclosed schematic diagram by the absence of capacitors in the forward signal paths in any of the electronic building blocks.

In an example, multiple polarity indicating outputs can be created using two polarized piezoelectric film sensors. Typically, the respiratory air temperature and pressure change transducers can be constructed of polyvinylidene (PVDF) film and can be included as part of a polarized respiratory air temperature and pressure change sensor. The adapter apparatus can contain a respiratory air temperature change signal-processing channel and a respiratory air pressure change signal-processing channel. The input section of the respiratory air temperature change signal-processing channel can consist of a differential amplifier and integrator with resistive reset coupled to receive the temperature change signal from the PVDF film transducer that makes up the sensing apparatus in the polarized respiratory air temperature and pressure change sensor. The input section of the respiratory air pressure change signal-processing channel can consist of a differential amplifier and integrator with resistive reset coupled to receive the pressure change signal from the PVDF film transducer that makes up the sensing apparatus in the polarized respiratory air temperature and pressure change sensor. Further, the signal processing channels can render the sensor outputs compatible with existing PSG machines.

In Example 1, an adapter apparatus for simultaneously processing respiratory air temperature information and respiratory air pressure information includes an electronic signal processing circuit configured to receive information indicative of respiratory air temperature from a respiratory air temperature piezoelectric film sensor and to receive information indicative of respiratory air pressure from a respiratory air pressure piezoelectric film sensor, wherein the electronic signal processing circuit is configured to simultaneously process the received respiratory air temperature information and the received respiratory air pressure information to produce a first signal output indicative of respiratory air temperature and a second signal output indicative of respiratory air pressure. The electronic signal processing circuit includes a first differential amplifier and signal integrator with resistive reset configured to average the received respiratory air temperature information to reduce differential noise and to attenuate common mode noise, and a second differential amplifier and signal integrator with resistive reset, separate from the first differential amplifier and signal integrator with resistive reset, configured to average the received respiratory air pressure information to reduce differential noise and to attenuate common mode noise.

In Example 2, the adapter apparatus of Example 1 optionally includes the respiratory air temperature piezoelectric film sensor and the respiratory air pressure piezoelectric film sensor.

In Example 3, the respiratory air temperature piezoelectric film sensor of any one or more of Examples 1-2 optionally includes a polyvinylidene fluoride film sensor configured to detect respiratory air temperature information.

In Example 4, the respiratory air pressure piezoelectric film sensor of any one or more of Examples 1-3 optionally includes a polyvinylidene fluoride film sensor configured to detect respiratory air pressure information.

In Example 5, the adapter apparatus of any one or more of Examples 1-4 optionally includes a single polyvinylidene fluoride film sensor configured to detect respiratory air temperature information and respiratory air pressure information, the single polyvinylidene fluoride film sensor including the respiratory air temperature piezoelectric film sensor and the respiratory air pressure piezoelectric film sensor.

In Example 6, the respiratory air temperature piezoelectric film sensor and the respiratory air pressure piezoelectric film sensor of any one or more of Examples 1-5 are optionally configured to provide a rigid phase and polarity relationship between respiratory air temperature and respiratory air pressure.

In Example 7, the first and second differential amplifiers and signal integrators with resistive resets of any one or more of Examples 1-6 are optionally configured to provide a predetermined gain factor by which the received respiratory air temperature information and the received respiratory air pressure information are amplified, to provide signal averaging over time to reduce differential noise, and to attenuate common-mode noise.

In Example 8, the electronic signal processing circuit of any one or more of Examples 1-7 optionally includes a first third order Butterworth low pass filters configured to remove components from the received respiratory air temperature information having a frequency above approximately 125 mHz, and a second third order Butterworth low pass filters configured to remove components from the received respiratory air pressure information having a frequency above approximately 1 Hz.

In Example 9, the first and second differential amplifiers and signal integrators with resistive resets of any one or more of Examples 1-8 are optionally configured to average the received respiratory air temperature information and the received respiratory air pressure information using a time constant based on a respiratory response time.

In Example 10, the first signal output indicative of respiratory air temperature of any one or more of Examples 1-9 optionally includes the averaged received respiratory air temperature information, and the second signal output indicative of respiratory air pressure includes the averaged received respiratory air pressure information.

In Example 11, the adapter apparatus of any one or more of Examples 1-10 optionally includes a cable configured to couple the integrated sensor to a PSG machine, wherein the electronic signal processing circuit is integrated with the cable.

In Example 12, a system includes a respiratory air temperature piezoelectric polyvinylidene fluoride film sensor configured to detect a respiratory air temperature of a subject, a respiratory air pressure piezoelectric polyvinylidene fluoride film sensor configured to detect a respiratory air pressure of the subject, an electronic signal processing circuit configured to receive information indicative of a respiratory air temperature from the respiratory air temperature piezoelectric polyvinylidene fluoride film sensor and information indicative of a respiratory air pressure from the respiratory air pressure piezoelectric polyvinylidene fluoride film sensor, wherein the electronic signal processing circuit is configured to simultaneously process the received respiratory air temperature information and the received respiratory air pressure information to produce a first signal output indicative of respiratory air temperature and a second signal output indicative of respiratory air pressure. The electronic signal processing includes a first differential amplifier and signal integrator with resistive reset configured to average the received respiratory air temperature information to reduce differential noise and to attenuate common mode noise, and a second differential amplifier and signal integrator with resistive reset, separate from the first differential amplifier and signal integrator with resistive reset, configured to average the received respiratory air pressure information to reduce differential noise and to attenuate common mode noise. The first signal output indicative of respiratory air temperature includes the averaged received respiratory air temperature information, and the second signal output indicative of respiratory air pressure includes the averaged received respiratory air pressure information. The system further includes a polysomnograph machine, coupled to the electronic signal processing circuit, the polysomnograph machine configured to receive the averaged received respiratory air temperature and the averaged received respiratory air pressure information from the electronic signal processing circuit and to provide the averaged received respiratory air temperature information and the averaged received respiratory air pressure information to a user.

In Example 13, the system of Example 12 optionally includes a single polyvinylidene fluoride film sensor configured to detect respiratory air temperature information and respiratory air pressure information, the single polyvinylidene fluoride film sensor including the respiratory air temperature piezoelectric polyvinylidene fluoride film sensor and the respiratory air pressure piezoelectric polyvinylidene fluoride film sensor.

In Example 14, a method for simultaneously processing respiratory air temperature information and respiratory air pressure information includes receiving information indicative of respiratory air temperature of a subject from a respiratory air temperature piezoelectric film sensor and information indicative of respiratory air pressure of the subject from a respiratory air pressure piezoelectric film sensor, and simultaneously processing the received respiratory air temperature information and the received respiratory air pressure information to produce a first electronic signal output indicative of respiratory air temperature and a second electronic signal output indicative of respiratory air pressure. The processing includes averaging the received respiratory air temperature information using a first differential amplifier and signal integrator with resistive reset to reduce differential noise and to attenuate common-mode noise, and averaging the received respiratory air pressure information using a second differential amplifier and signal integrator with resistive reset, separate from the first differential amplifier and signal integrator with resistive reset, to reduce differential noise and to attenuate common-mode noise.

In Example 15, the receiving the respiratory air temperature information of Example 14 optionally includes using a respiratory air temperature piezoelectric polyvinylidene fluoride film sensor, and the receiving the respiratory air pressure information include using a respiratory air pressure piezoelectric polyvinylidene fluoride film sensor.

In Example 16, the receiving the respiratory air temperature information and the receiving the respiratory air pressure information of any one or more of Examples 14-15 optionally includes using a single polyvinylidene fluoride film sensor configured to detect respiratory air temperature information and respiratory air pressure information.

In Example 17, the receiving the respiratory air temperature information and the respiratory air pressure information of any one or more of Examples 14-16 optionally includes receiving rigid phase and polarity relationship information between respiratory air temperature and respiratory air pressure.

In Example 18, the simultaneously processing the received respiratory air temperature information and the received respiratory air pressure information of any one or more of Examples 14-17 optionally includes amplifying the received respiratory air temperature information and the received respiratory air pressure information by a predetermined gain factor using the first and second differential amplifiers and signal integrators with resistive resets, to provide signal averaging over time to reduce differential noise, and to attenuate common-mode noise.

In Example 19, the simultaneously processing the received respiratory air temperature information and the received respiratory air pressure information of any one or more of Examples 14-18 optionally includes removing components from the received respiratory air temperature information having a frequency above approximately 125 mHz using a first third order Butterworth low pass filter, and removing components from the received respiratory air pressure information having a frequency above approximately 1 Hz using a second third order Butterworth low pass filter.

In Example 20, the averaging the received respiratory air temperature information and the received respiratory air pressure information of any one or more of Examples 14-19 optionally includes using a time constant based on a respiratory response time of the subject.

Further areas of applicability will become apparent from the description provided herein. It should be understood that the description and specific examples are intended for purposes of illustration only and are not intended to limit the scope of the present disclosure.

### DESCRIPTION OF THE DRAWINGS

The drawings described herein are for illustration purposes only and are not intended to limit the scope of the present disclosure in any way.

The forgoing features, objects and advantages of the invention will become apparent to those skilled in the art from the following detailed description of a preferred embodiment, especially when considered in conjunction with the accompanying drawings in which like the numerals in the several views refer to the corresponding parts:

FIGS. 1-3 illustrate generally an example of a system including an adapter apparatus for processing respiratory air temperature and pressure change transducer signals.

FIG. 4 illustrates generally an example of a schematic diagram of an adapter apparatus for processing respiratory air temperature and pressure change transducer signals.

### DETAILED DESCRIPTION

The following description is merely exemplary in nature and is not intended to limit the present disclosure, application, or uses. It should be understood that throughout the drawings, corresponding reference numerals indicate like or corresponding parts and features.

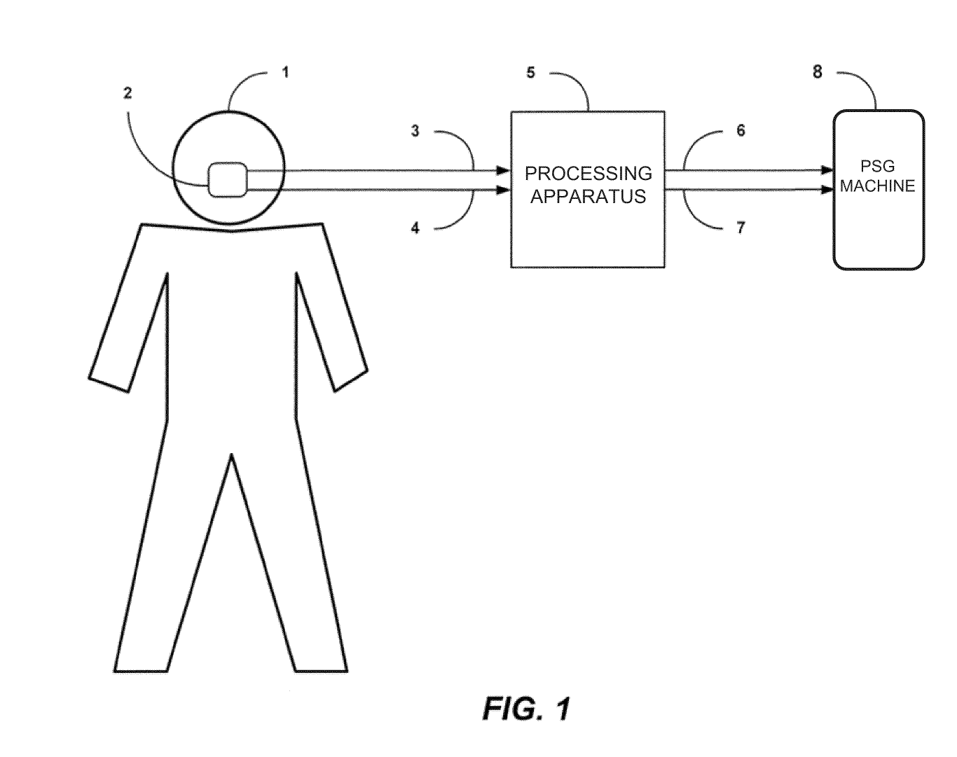

FIG. 1 illustrates generally an example of a system including an adapter apparatus 5 for processing respiratory air temperature and pressure change transducer signals. Referring to FIG. 1, there is indicated generally by numeral 1 a typical sleep laboratory patient who has been outfitted with a respiratory air temperature and pressure change sensor 2 to measure respiratory air flow. A pair of temperature signal leads 3 and a pair of pressure signal leads 4 connect the respiratory air temperature and pressure change sensor 2 to the adapter apparatus 5 for processing respiratory air temperature and pressure change transducer signals.

This specific exemplary embodiment shows two polarity indicating output wire pairs 6 and 7 connecting the adapter apparatus 5 for processing respiratory air temperature and pressure change transducer signals and presenting same to a conventional, commercially-available PSG machine 8, such as:

Model Sandman available from Covidien of Kanata, ON Canada;

Model Alice available from Respironics Inc of Murrysville, Pa.;

Model Connex available from Natus Medical of Oakville, ON Canada;

Model Harmonie-S available from Stellate of Montreal, QC Canada;

Model Polysmith available from Nihon Kohden America of Foothill Ranch, Calif.;

Model Comet available from Astro-Med, Inc of West Warwick, R.I.;

Model Embletta available from Embla of Broomfield, Colo.;

Model E Series available from Compumedics of Charlotte, N.C.;

Model 20B available from CleveMed of Cleveland, Ohio;

Model Somnostar available from Cardinal Health of Yorba Linda, Calif.;

Model Easy II available from Cadwell Laboratories, Inc of Kennewick, Wash.;

Model Pursuit Sleep2 available from Braebon of Ogdensburg, N.Y.; or

Model SleepScan available from Natus Medical of Mundelein, Ill.

All trademarks are property of their respective owners. This list is only exemplary in nature and does not claim to be comprehensive or complete.

In a typical sleep laboratory application, temperature signal output 6 is configured to produce the respiratory air temperature change showing inhalation as an upward deflection of respiratory effort and showing exhalation as a downward deflection of respiratory effort on the PSG machine 8 display.

Furthermore, pressure signal output 7 is configured to produce the respiratory air pressure change showing inhalation as an upward deflection of respiratory effort and showing exhalation as a downward deflection of respiratory effort on the PSG machine 8 display.

It is by international convention and by requirement of the American Association of Sleep Medicine (AASM) that a patient's inhalation produces an upward deflection and exhalation produces a downward deflection on the PSG machine 8 display.

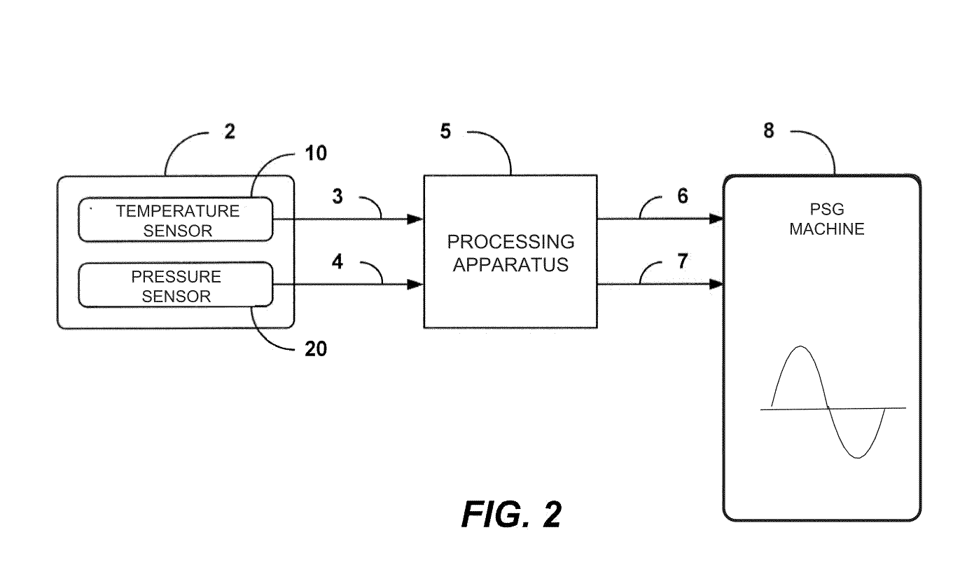

FIG. 2 illustrates generally an example of a system including an adapter apparatus 5 for processing respiratory air temperature and pressure change transducer signals, including an example of wiring connections connecting the adapter apparatus 5 to the respiratory air temperature and pressure change sensor 2. In an example, the respiratory air temperature and pressure change sensor 2 can include a temperature change sensing PVDF film transducer 10 and a pressure change sensing PVDF film transducer 20. A pair of temperature signal leads 3 and a pair of pressure signal leads 4 connect the respiratory air temperature and pressure change sensor 2 to the adapter apparatus 5 for processing respiratory air temperature and pressure change transducer signals.

This specific exemplary of the embodiment shows two polarity indicating output wire pairs 6 and 7 connecting the adapter apparatus 5 for processing respiratory air temperature and pressure change transducer signals to a conventional, commercially-available PSG machine 8.

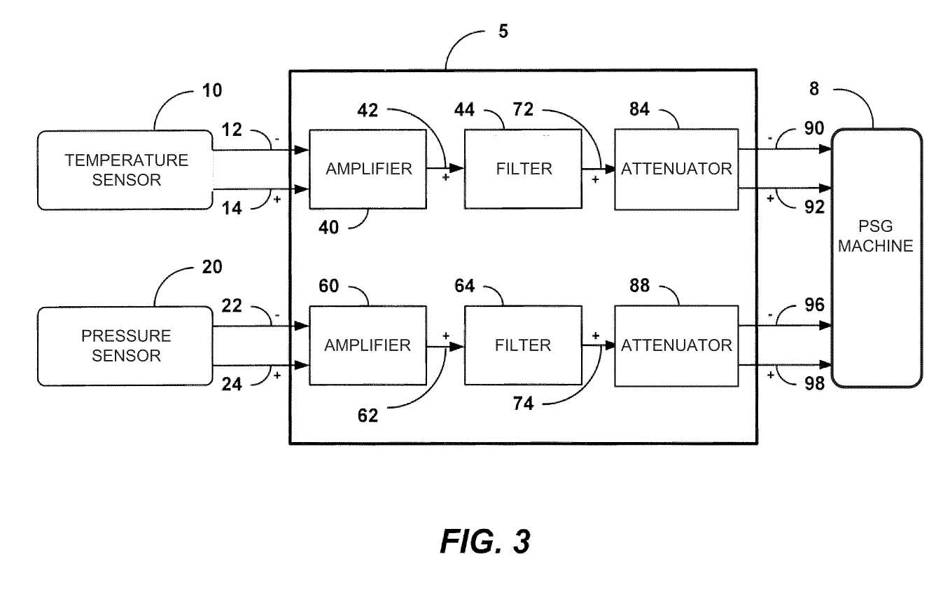

FIG. 3 illustrates generally an example of a system including an adapter apparatus 5 for processing respiratory air temperature and pressure change transducer signals, including an example of functional components included in the adapter apparatus 5.

The temperature change sensing PVDF film transducer 10 in one example is constructed in accordance with the teachings of the commonly-assigned Reinhold Henke et al. U.S. Provisional Application 61/075,124, entitled “Polarized Respiratory Air Temperature and Pressure Change Sensor,” filed on Jun. 24, 2008, incorporated herein in its entirety. The sensor 10 is adapted to sense a patient's inspiratory and expiratory air temperature.

In an example, the temperature change sensing PVDF film transducer 10 connects to the respiratory air temperature signal path differential amplifier and integrator with resistive reset 40 via a pair of input wire leads 2-14.

In this example, wire 12 of the input wire pair is indicated to represent the positive terminal of the temperature change sensing PVDF film transducer that goes positive when patient is inhaling. Further, wire 14 of the input wire pair is indicated to represent the negative terminal of the temperature change sensing PVDF film transducer that goes positive when patient is inhaling. The differential amplifier and integrator with resistive reset 40 of the respiratory air temperature change signal path comprises a differential type amplifier which functions to increase the common-mode rejection of the adapter apparatus 5 so as to make it less susceptible to 60 Hz noise present in the environment as well as to motion artifacts. The signal integrator with resistive reset serves to slowly average the incoming signal over time so that the differential amplifier only amplifies signals that are within the response time of interest, i.e., the patient's respiratory response time. In various examples, the averaging signal integrator may operate with a fixed time constant of about 62.5 ms.

Without limitation, the differential amplifier and integrator with resistive reset 40 may have a gain in the range of from 2 to 10 with about 6.2 being quite adequate.

The output signal from the differential input amplifier and integrator with resistive reset 40 on lead 42 is applied to a 3^{rd}-order Butterworth low pass filter 44. It should be understood by those skilled in the art that the type of filter response is neither limited to a 3^{rd}-order filter nor is it limited to a Butterworth response. Other filter responses may also be used.

Typically, but not limited to, the cut-off frequency for the third order Butterworth low pass filter 44 may be about 125 mHz.

As seen in the example of FIG. 2, the output 72 of the respiratory air temperature change signal path low pass filter 44 can connect to an input of the respiratory air temperature change signal path output attenuator module 84.

In an example, the respiratory air temperature change signal path output attenuator 84 attenuates the signal coming from the respiratory air temperature change signal path low pass filter module 44 in order to reduce the temperature change signal path amplitude to a level that is compliant with the requirements of the input specifications of the input jack of the particular PSG machine 8 employed by way of lines 90 and 92 respectively.

It should be clear to those skilled in the art that the entire temperature signal path starting from the polarized piezoelectric film sensor 10 and ending at the PSG machine 8 is DC coupled, thus ensuring that the relationship of polarized piezoelectric film sensor polarity and indication of respiration effort between inhalation and exhalation on the PSG machine is purposely maintained.

The pressure change sensing PVDF film transducer 20 is in some examples is constructed in accordance with the teachings of the afore-referenced patent application of Reinhold Henke and entitled “Polarized Respiratory Air Temperature and Pressure Change Sensor”. The sensor 20 is adapted to sense a sleep lab subject's inspiratory and expiratory air pressure.

The pressure change sensing PVDF film transducer 20 connects to the respiratory air pressure signal path differential amplifier and integrator with resistive reset 60 via a pair of input wire leads 22-24.

Wire 22 of the input wire pair is indicated to represent the positive terminal of the pressure change sensing PVDF film transducer that goes positive when patient is inhaling.

Wire 24 of the input wire pair is indicated to represent the negative terminal of the pressure change sensing PVDF film transducer that goes positive when patient is inhaling. The differential amplifier and integrator with resistive reset 60 of the respiratory air pressure change signal path comprises a differential type amplifier which functions to increase the common-mode rejection of the adapter apparatus 5 so as to make it less susceptible to 60 Hz noise present in the environment as well as to motion artifacts. The signal integrator with resistive reset serves to slowly average the incoming signal over time so that the differential amplifier only amplifies signals that are within the response time of interest, i.e., the patient's respiratory response time. In various examples, the averaging signal integrator may operate with a fixed time constant of about 62.5 ms.

Without limitation, the differential amplifier and integrator with resistive reset 60 may have a gain in the range of from 2 to 10 with about 6.2 being adequate.

The output on wire 62 from the differential input amplifier and integrator with resistive reset 60 is applied to a third order Butterworth low pass filter 64. The input of the respiratory air pressure change signal path low pass third order Butterworth filter 64 is connected to the output wire 62 of the differential input amplifier 60.

It should be understood by those skilled in the art that the type of filter response is neither limited to a third order filter nor is it limited to a Butterworth response. Other filter responses may also be used.

Typically, the cut-off frequency for the third order Butterworth low pass filter 64 may be about 1 Hz, but limitation thereto is not intended.

The output on line 74 of the respiratory air pressure change signal path low pass filer 3rd order Butterworth filter module 64 connects to the input of the respiratory air pressure change signal path output attenuator module 88.

The respiratory air pressure change signal path output attenuator 88 functions to attenuate the signal coming from the respiratory air pressure change signal low pass filter 3rd order Butterworth filter module 64 in order to reduce the pressure change signal path amplitude to a level that is compliant with the requirements of the input specifications of the input jack of the PSG machine 8 by way of lines 96 and 98 respectively.

It should be clear to those skilled in the art that the entire pressure signal path starting from the polarized piezoelectric film sensor 20 and ending at the PSG machine 8 is DC coupled, thus ensuring that the relationship of polarized piezoelectric film sensor polarity and indication of respiration effort between inhalation and exhalation on the PSG machine is purposely maintained.

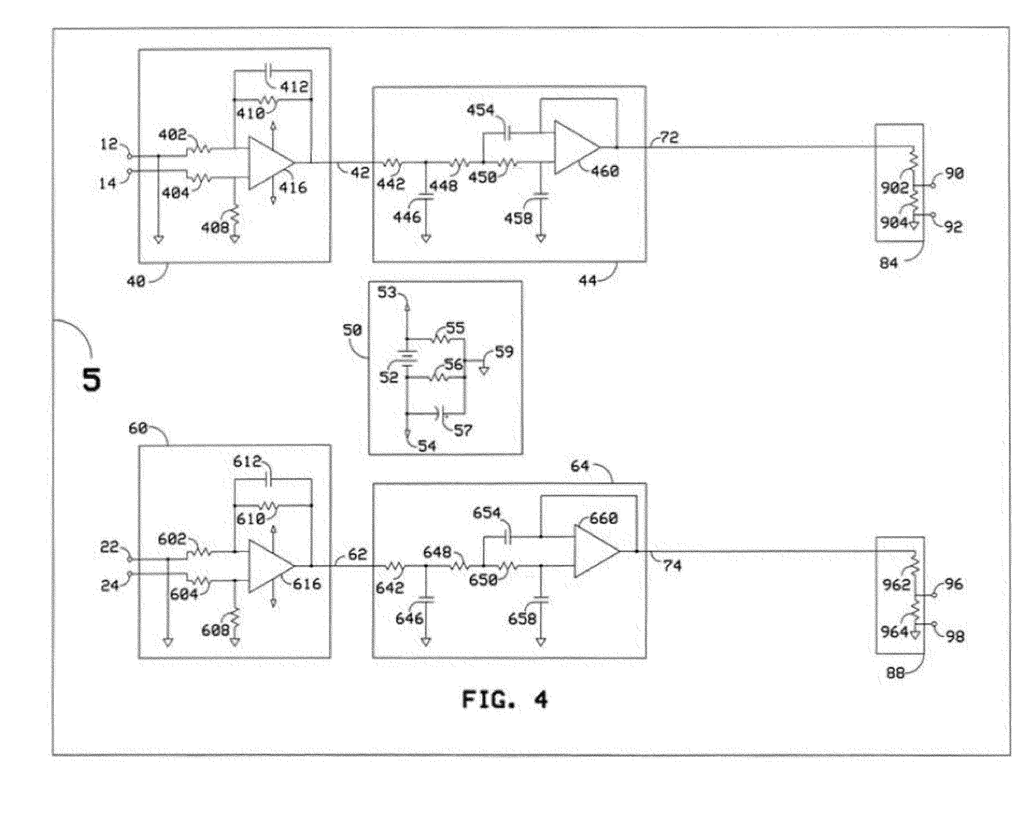

Having described an example overall configuration of the adapter apparatus of FIG. 3, a more detailed explanation of a specific implementation of the adapter will now be presented and, in that regard, reference is made to the schematic circuit diagram of FIG. 4.

FIG. 4 illustrates generally an example of a schematic diagram of an adapter apparatus 5 for processing respiratory air temperature and pressure change transducer signals.

In an example, the adapter apparatus 5 can be integrated with the cable used to couple the temperature change and pressure change sensing PVDF film transducers 10 and 20, respectively, to the polysomnograph machine 8. As such, it incorporates its own power supply and virtual ground generator 50 in the form of a single lithium battery 52 with its positive battery voltage terminal 53 identified as V+and its negative battery voltage terminal 54 labeled V-. A resistor 55 connects the positive battery voltage terminal to a virtual ground point 59. A resistor 56 connects the negative battery voltage terminal to the virtual ground point 59. Resistors 55 and 56 are preferably of equal value in establishing virtual ground point 59. A polarized capacitor 57 connects in parallel with resistors 56 to form a low alternating current (AC) impedance return path from the negative battery terminal 54 to the virtual ground point 59.

The input terminal 12 to the differential amplifier and integrator with resistive reset 40 is coupled, via resistor 402 to the inverting input of operational amplifier 416, to the gain setting and integrator resetting resistor 410 and to the integrating capacitor 412. The input terminal 14 connects to the non-inverting input of differential operational amplifier and integrator with resistive reset 416, via a resistor 404 and to an input load resistor 408.

The output from the differential input amplifier circuit 416 appears on lead 42 and connects to the respiratory air temperature change signal path third order Butterworth low-pass filter circuit 44.

Referring to filter circuit 44, the input appearing on lead 42 is applied, via series connected resistors 442, 448 and 450, to the non-inverting input of an operational amplifier 460 and those resistors, along with capacitors 446, 454 and 458 cooperate with the operational amplifier 460 to function as a low-pass Butterworth filter. The output of the operational amplifier 460 is presented to lead 72.

The values of the resistors 442, 448 and 450 and the capacitors 446, 454 and 458 may be set to establish a cut-off frequency of the third order Butterworth low-pass filter circuit 44 to about 125 mHz as mentioned previously.

Lead 72 feeds into the respiratory air temperature change signal path output attenuator 84, which comprises a voltage divider including resistors 902 and 904 to drop the polarized piezoelectric film sensor based signal component to acceptable levels of the PSG machine to which the polarized piezoelectric film sensor is being interfaced via a pair of lead wires 90 and 92, respectively.

The input terminal 22 of the differential amplifier and integrator with resistive reset 60 is coupled, via resistor 602 to the inverting input of operational amplifier 616, to the gain setting and integrator-resetting resistor 610 and to the integrating capacitor 612. The input terminal 24 of the differential amplifier and integrator with resistive reset 60 is coupled, via resistor 604 to the non-inverting input of operational amplifier 616 and to the input load resistor 608.

The output from the differential input amplifier circuit 616 appears on lead 62 and connects to the respiratory air pressure signal path third order Butterworth low-pass filter circuit 64.

Referring to filter circuit 64, the input appearing on lead 62 is applied, via series connected resistors 642, 654 and 650, to the non-inverting input of an operational amplifier 660 and those resistors, along with capacitors 646, 652 and 658 cooperate with the operational amplifier 660 to function as a low-pass filter. The output of the operational amplifier 660 is presented on lead 74.

The values of the resistors 642, 648 and 650 and the capacitors 646, 654 and 658 may be set to establish a cut-off frequency of the third order Butterworth low-pass filter circuit 64 to about 1 Hz as mentioned previously.

Lead 74 feeds into the respiratory air pressure change signal path output attenuator 88, which comprises a voltage divider including resistors 962 and 964 to drop the polarized piezoelectric film sensor based signal component to acceptable levels of the PSG machine to which the polarized piezoelectric film sensor is being interfaced via a pair of lead wires 96 and 98, respectively.

The list of specific components used to assemble a printed circuit board assembly is known in the industry as a Bill-of-Materials (BOM). Below is an example of a BOM for one embodiment of the components of FIG. 4.

| | |
|---|---|
| B1 | BR2330A/FA |
| C1 | 0.1 uF |
| C3 | 1 uF |
| C5 | 0.39 uF |
| C6 | 0.056 uF |
| C7 | 0.1 uF |
| C8 | 1 uF |
| C9 | 0.39 uF |
| C10 | 0.056 uF |
| C11 | 10 uF/tant |
| R1 | 1.00 M |
| R2 | 100k |
| R3 | 100k |
| R4 | 10.0k |
| R5 | 560k |
| R6 | 560k |
| R7 | 560k |
| R9 | 1.00k |
| R11 | 2.7 M |
| R16 | 100k |
| R18 | 10.0k |
| R19 | 1.00 M |
| R20 | 3.30 M |
| R21 | 3.30 M |
| R22 | 3.30 M |
| R23 | 1.00k |
| R24 | 100k |
| R25 | 2.70 M |
| R26 | 330k |
| R27 | 330k |
| U1:A | LMC6442AIM |
| U1:B | LMC6442AIM |
| U2:A | LMC6442AIM |
| U2:B | LMC6442AIM |

During operation in a typical application, such as in a sleep laboratory, a patient is fitted with a polarized respiratory air temperature and pressure change sensor. In an example, the polarized respiratory air temperature and pressure change sensor can be connected using an adapter apparatus to a PSG machine. The adapter apparatus has been described herein for sleep scientists, sleep physicians and sleep technicians to see, detect and properly diagnose specific sleep disorders and diseases which including abnormal respiratory events including events occurring in the upper airway of the patient.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

What is claimed is:

1. An adapter apparatus for simultaneously processing respiratory air temperature information and respiratory air pressure information, comprising:
an electronic signal processing circuit configured to receive information indicative of respiratory air temperature from a respiratory air temperature piezoelectric film sensor and to receive information indicative of respiratory air pressure from a respiratory air pressure piezoelectric film sensor, wherein the electronic signal processing circuit is configured to simultaneously process the received respiratory air temperature information and the received respiratory air pressure information to produce a first signal output indicative of respiratory air temperature and a second signal output indicative of respiratory air pressure, the electronic signal processing circuit including:
a first differential amplifier and signal integrator with resistive reset configured to average the received respiratory air temperature information to reduce differential noise and to attenuate common mode noise; and
a second differential amplifier and signal integrator with resistive reset, separate from the first differential amplifier and signal integrator with resistive reset, configured to average the received respiratory air pressure information to reduce differential noise and to attenuate common mode noise.

2. The adapter apparatus of claim 1, including the respiratory air temperature piezoelectric film sensor and the respiratory air pressure piezoelectric film sensor.

3. The adapter apparatus of claim 1, wherein the respiratory air temperature piezoelectric film sensor includes a polyvinylidene fluoride film sensor configured to detect respiratory air temperature information.

4. The adapter apparatus of claim 1, wherein the respiratory air pressure piezoelectric film sensor includes a polyvinylidene fluoride film sensor configured to detect respiratory air pressure information.

5. The adapter apparatus of claim 1, including a single polyvinylidene fluoride film sensor configured to detect respiratory air temperature information and respiratory air pressure information, the single polyvinylidene fluoride film sensor including the respiratory air temperature piezoelectric film sensor and the respiratory air pressure piezoelectric film sensor.

6. The adapter apparatus of claim 1, wherein the respiratory air temperature piezoelectric film sensor and the respiratory air pressure piezoelectric film sensor are configured to provide a rigid phase and polarity relationship between respiratory air temperature and respiratory air pressure.

7. The adapter apparatus of claim 1, wherein the first and second differential amplifiers and signal integrators with resistive resets are configured to provide a predetermined gain factor by which the received respiratory air temperature information and the received respiratory air pressure information are amplified, to provide signal averaging over time to reduce differential noise, and to attenuate common-mode noise.

8. The adapter apparatus of claim 1, wherein the electronic signal processing circuit includes:
a first third order Butterworth low pass filters configured to remove components from the received respiratory air temperature information having a frequency above approximately 125 mHz; and
a second third order Butterworth low pass filters configured to remove components from the received respiratory air pressure information having a frequency above approximately 1 Hz.

9. The adapter apparatus of claim 1, wherein the first and second differential amplifiers and signal integrators with resistive resets are configured to average the received respiratory air temperature information and the received respiratory air pressure information using a time constant based on a respiratory response time.

10. The adapter apparatus of claim 1, wherein the first signal output indicative of respiratory air temperature includes the averaged received respiratory air temperature information, and the second signal output indicative of respiratory air pressure includes the averaged received respiratory air pressure information.

11. The adapter apparatus of claim 1, including a cable configured to couple the integrated sensor to a PSG machine, wherein the electronic signal processing circuit is integrated with the cable.

12. A system, comprising:
a respiratory air temperature piezoelectric polyvinylidene fluoride film sensor configured to detect a respiratory air temperature of a subject;
a respiratory air pressure piezoelectric polyvinylidene fluoride film sensor configured to detect a respiratory air pressure of the subject;
an electronic signal processing circuit configured to receive information indicative of a respiratory air temperature from the respiratory air temperature piezoelectric polyvinylidene fluoride film sensor and information indicative of a respiratory air pressure from the respiratory air pressure piezoelectric polyvinylidene fluoride film sensor, wherein the electronic signal processing circuit is configured to simultaneously process the received respiratory air temperature information and the received respiratory air pressure information to produce a first signal output indicative of respiratory air temperature and a second signal output indicative of respiratory air pressure, the electronic signal processing including:
a first differential amplifier and signal integrator with resistive reset configured to average the received respiratory air temperature information to reduce differential noise and to attenuate common mode noise; and
a second differential amplifier and signal integrator with resistive reset, separate from the first differential amplifier and signal integrator with resistive reset, configured to average the received respiratory air pressure information to reduce differential noise and to attenuate common mode noise,
wherein the first signal output indicative of respiratory air temperature includes the averaged received respiratory air temperature information, and the second signal output indicative of respiratory air pressure includes the averaged received respiratory air pressure information; and
a polysomnograph machine, coupled to the electronic signal processing circuit, the polysomnograph machine configured to receive the averaged received respiratory air temperature and the averaged received respiratory air pressure information from the electronic signal processing circuit and to provide the averaged received respiratory air temperature information and the averaged received respiratory air pressure information to a user.

13. The system of claim 12, including a single polyvinylidene fluoride film sensor configured to detect respiratory air temperature information and respiratory air pressure information, the single polyvinylidene fluoride film sensor including the respiratory air temperature piezoelectric polyvinylidene fluoride film sensor and the respiratory air pressure piezoelectric polyvinylidene fluoride film sensor.

14. A method for simultaneously processing respiratory air temperature information and respiratory air pressure information, comprising:
receiving information indicative of respiratory air temperature of a subject from a respiratory air temperature piezoelectric film sensor and information indicative of respiratory air pressure of the subject from a respiratory air pressure piezoelectric film sensor; and
simultaneously processing the received respiratory air temperature information and the received respiratory air pressure information to produce a first electronic signal output indicative of respiratory air temperature and a second electronic signal output indicative of respiratory air pressure, the processing including:
averaging the received respiratory air temperature information using a first differential amplifier and signal integrator with resistive reset to reduce differential noise and to attenuate common-mode noise; and
averaging the received respiratory air pressure information using a second differential amplifier and signal integrator with resistive reset, separate from the first differential amplifier and signal integrator with resistive reset, to reduce differential noise and to attenuate common-mode noise.

15. The method of claim 14, wherein the receiving the respiratory air temperature information includes using a respiratory air temperature piezoelectric polyvinylidene fluoride film sensor, and the receiving the respiratory air pressure information include using a respiratory air pressure piezoelectric polyvinylidene fluoride film sensor.

16. The method of claim 14, wherein the receiving the respiratory air temperature information and the receiving the respiratory air pressure information includes using a single polyvinylidene fluoride film sensor configured to detect respiratory air temperature information and respiratory air pressure information.

17. The method of claim 14, wherein the receiving the respiratory air temperature information and the respiratory air pressure information includes receiving rigid phase and polarity relationship information between respiratory air temperature and respiratory air pressure.

18. The method of claim 14, wherein the simultaneously processing the received respiratory air temperature information and the received respiratory air pressure information includes amplifying the received respiratory air temperature information and the received respiratory air pressure information by a predetermined gain factor using the first and second differential amplifiers and signal integrators with resistive resets, to provide signal averaging over time to reduce differential noise, and to attenuate common-mode noise.

19. The method of claim 14, wherein the simultaneously processing the received respiratory air temperature information and the received respiratory air pressure information includes:
removing components from the received respiratory air temperature information having a frequency above approximately 125 mHz using a first third order Butterworth low pass filter; and
removing components from the received respiratory air pressure information having a frequency above approximately 1 Hz using a second third order Butterworth low pass filter.

20. The method of claim 14, wherein the averaging the received respiratory air temperature information and the received respiratory air pressure information includes using a time constant based on a respiratory response time of the subject.
